(19) United States
(12) Patent Application Publication
(10) Pub. No.: US 2026/0263835 A1
(43) Pub. Date: Sep. 10, 2026

(54) RADIATION TREATMENT PLAN OPTIMIZATION METHOD AND APPARATUS

(71) Applicant: Siemens Healthineers International AG, Steinhausen (CH)

(72) Inventors: Martin Kraus, Fürth (DE); Florin-Cristian Ghesu, Baiersdorf (DE); Esa Kuusela, Espoo (FI)

(21) Appl. No.: 19/073,564

(22) Filed: Mar. 7, 2025

(51) Int. Cl. A61N 5/10

(52) U.S. Cl. CPC A61N 5/1031; A61N 5/1038; A61N 5/1064

(57) ABSTRACT

A control circuit accesses at least one objective function to be optimized pursuant to radiation treatment planning via an iterative optimization process comprising a plurality of iteration rounds where, in at least some of the iteration rounds, the control circuit employs a neural network that outputs at least one corresponding fluence map before ultimately outputting an optimized radiation treatment plan.

### TECHNICAL FIELD

These teachings relate generally to treating a patient's planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

### BRIEF DESCRIPTION OF DRAWINGS

The above needs are at least partially met through provision of described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings; and

FIG. 3 comprises a visualization diagram as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, these various embodiments can facilitate optimizing a radiation treatment plan for a particular patient using a particular radiation treatment apparatus where the radiation treatment plan comprises a control point sequence. By one approach, a control circuit accesses at least one objective function to be optimized via an iterative optimization process comprising a plurality of iteration rounds where, in at least some of the iteration rounds, the control circuit employs a neural network that outputs at least one corresponding fluence map before ultimately outputting an optimized radiation treatment plan.

By one approach, at least some of the aforementioned iteration rounds each comprise sequencing only a single field. By another approach, at least some of the iteration rounds each comprise sequencing at least two fields. By yet another approach, at least one of the iteration rounds comprises sequencing at least two fields while holding static at least one field.

By one approach, at least some of the aforementioned iteration rounds comprise accessing a current fluence map, computing radiation dose as a function of that current fluence map, computing dose objectives and an at least approximate derivative of the at least one objective function with respect to the current fluence map, generating a new target fluence map as a function of at least one previous fluence map and the at least approximate derivative of the at least one objective function with respect to the current fluence map, and then generating a new fluence map as a projection of the new target fluence map, wherein the new fluence map serves as the current fluence map in a subsequent iteration round.

By one approach, these teachings will accommodate training the aforementioned neural network using a plurality of input/output pairs. By one approach, the input/output pairs include at least one of historical optimization trajectory information, automatically generated optimization trajectory information, and/or automatically generated fluence maps and corresponding sequenceable control points.

By one approach, these teachings will also accommodate generating a plurality of fluence maps and then identifying at least one of the plurality of fluence maps having a predetermined acceptable level of sequenceability to provide a selected fluence map and using that selected fluence map as training target for the aforementioned neural network. By one approach, the aforementioned acceptable level of sequenceability corresponds to an objective measure of sequencing error.

By one approach, these teachings can comprise a computer program that itself comprises instructions that, when the computer program is executed by a computer, causes the computer to carry out any or all of the steps, actions, and/or functions described herein, including accessing at least one objective function to be optimized via an iterative optimization process comprising a plurality of iteration rounds where, in at least some of the iteration rounds, the computer program employs a neural network that outputs at least one corresponding fluence map before ultimately outputting an optimized radiation treatment plan.

So configured, these teachings allow use of computation approaches that are quickly computed. That savings in time, in turn, allows optimization loops to be quickly completed, thereby greatly shortening the amount of time required to optimize a radiation treatment plan.

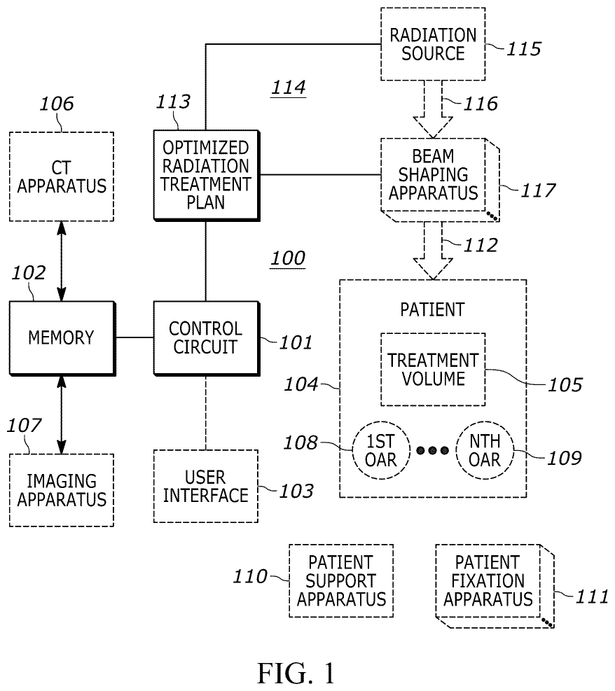

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be appreciated that the control circuit 101 may comprise a single integrated platform or may comprise a plurality of such circuits that work in cooperation with one another.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101). As with the control circuit 101, the memory 102 may comprise a singular structure or may comprise a plurality of memory platforms that collectively comprise the “memory” of this apparatus 100.

In addition to information such as optimization information for a particular patient and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

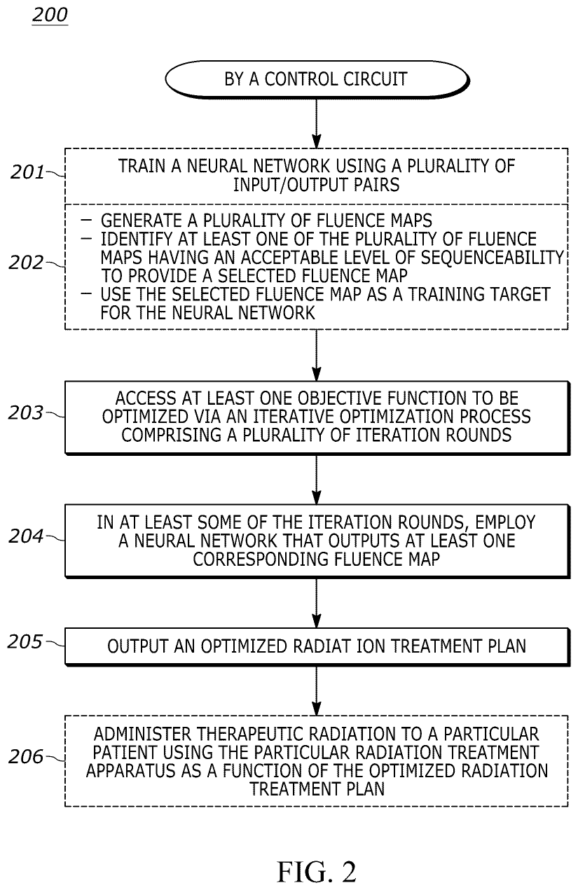

Referring now to FIG. 2, a process 200 that can be carried out, for example, in conjunction with the above-described application setting (and more particularly via the aforementioned control circuit 101) will be described. Generally speaking, this process 200 serves to facilitate generating an optimized radiation treatment plan 113 to thereby facilitate treating a particular patient with therapeutic radiation using a particular radiation treatment platform per that optimized radiation treatment plan.

Generally speaking, this process 200 serves to optimize a radiation treatment plan for a particular patient using a particular radiation treatment apparatus, where the radiation treatment plan itself comprises a control point sequence. In radiation treatment planning, particularly for techniques such as Intensity-Modulated Radiation Therapy (IMRT) and Volumetric Modulated Arc Therapy (VMAT), a control point sequence is a series of discrete points along a treatment beam's path where the radiation delivery parameters are specified and can be adjusted. Each control point represents a specific moment in time during the delivery of the radiation dose, and the parameters defined at these points often include the shape of the radiation beam (often defined by multi-leaf collimator positions), the gantry angle, the dose rate, and/or the cumulative monitor units (MUs). The sequence of control points is used by the radiation source to modulate the intensity and shape of the radiation beam as it moves around or across the patient, enabling the sculpting of the dose distribution to conform to the target volume while sparing adjacent healthy tissues.

As will be described below, this process 200 makes use of a neural network. Optional block 201 presents an approach to training this neural network using a plurality of input/output pairs. Training a neural network using input/output pairs typically involves presenting the network with a series of examples, where each example consists of a known input and its corresponding desired output. The network processes the inputs through interconnected layers of neurons, each performing a simple computation, to produce its own outputs. The discrepancy between the network's outputs and the desired outputs can then be quantified using a loss function. This loss is backpropagated through the network, informing an optimization algorithm (such as gradient descent) how to adjust the weights and biases of the neurons to minimize the loss. Over many iterations of this process, involving potentially vast numbers of input/output pairs, the network learns the underlying patterns that map inputs to outputs, thereby improving its ability to make accurate predictions or decisions when presented with new, unseen data.

By one approach, the aforementioned input/output pairs can include at least one of, and potentially all of, historical optimization trajectory information, automatically generated optimization trajectory information, and/or automatically generated fluence maps and corresponding sequenceable control points. (A fluence map is a representation of the distribution and intensity of the radiation dose that is to be delivered to a given treatment area. By one approach, a fluence map describes the number of radiation particles or photons (measured in particles per unit area) that are intended to be delivered across the treatment field. Such a map is sometimes displayed as a grid or matrix with each element representing a specific location in the treatment field and the value within that element indicating the relative fluence of radiation at that point.)

As presented at optional block 202, the foregoing training can also include, if desired, generating a plurality of fluence maps, identifying at least one of the plurality of fluence maps having an acceptable level of sequenceability to provide a selected fluence map (which acceptable level of sequenceability can correspond, for example, to an objective measure of sequencing error as established and selected by the user), and then using the selected fluence map as a training target for the neural network.

At block 203, the control circuit 101 accesses at least one objective function to be optimized via an iterative optimization process comprising a plurality of iteration rounds. During this iterative optimization process, by one approach, at least some of the iteration rounds each comprise sequencing only a single field. By one approach, in lieu of the foregoing or in combination therewith, at least some of the iteration rounds each comprise sequencing at least two fields. And by yet another approach, and again in lieu of the foregoing or in combination therewith, at least one of the iteration rounds comprises sequencing at least two fields while holding static at least one field.

These teachings are highly flexible in practice and will accommodate various supplemental activity and/or modified activity. As one example in these regards, by one approach at least some of the iteration rounds can comprise accessing a current fluence map and then computing radiation dose as a function of the current fluence map. The control circuit 101 can then compute dose objectives and an at least approximate derivative of the at least one objective function with respect to the current fluence map, and then generate a new target fluence map as a function of at least one previous fluence map and the at least approximate derivative of the at least one objective function with respect to the current fluence map. So configured, the control circuit 101 can then generate a new fluence map as a projection of the new target fluence map, wherein the new fluence map serves as the current fluence map in a subsequent iteration round.

As shown in block 204, this process 200 also provides that in at least some of the iteration rounds, the process 200 will employ a neural network that outputs at least one corresponding fluence map. Further exemplary details in these regards appear below.

At block 205 the control circuit 101 outputs an optimized radiation treatment plan as a result of the foregoing activity and at optional block 206 this process 200 will accommodate administering therapeutic radiation to a particular patient using the particular radiation treatment apparatus as a function of the optimized radiation treatment plan.

Further details that comport with these teachings will now be presented. It will be understood that the specific details of these examples are intended to serve an illustrative purpose and are not intended to suggest any particular limitations with respect to these teachings.

It may be helpful to first recall that modern radiation treatment plan creation is typically based on an optimizer that can create a machine control point sequence by minimizing a user-specified objective function. The machine control point sequence often includes the specifications of all machine axis in multiple consecutive time instances. Most of the axis are related to individual leaf positions of a multi-leaf-collimator. The optimization usually involves iterative gradient-based updates of multi-leaf collimator positions and so forth, trying to optimize an objective function with respect to dose distributions in regions of interest of the patient's body. The details of the optimizer depends on which kind of plan the user is requesting, but at the moment, it is common to request treatment planning that would deliver dose and modulate the leaves while the gantry is simultaneously rotating (a volumetric modulated arc therapy approach (VMAT)). One commonly used approach in VMAT optimization is to divide the arc-beam(s) into sectors and associate fluence maps for those fields and then do the multi-leaf collimator leaf position optimization by first determining the target values for those fluence maps, leading, for example, to the following optimization loop:
(A) Initial/Current multi-leaf collimator positions
(B) Create Fluence Map(s)
(D) Compute Dose based on Fluence Map(s)
(E) Compute Dose Objectives and the (approximate) derivative of the objective function with respect to the fluence map(s)
(F) Create a new target fluence map(s) based on the derivative and previous map(s)
(G) Use a Sequencing Module to map from target fluences to new multi-leaf collimator positions
(H) Repeat

Following such an approach, plan optimization is a time-consuming process. That reality, in turn, limits how many plans can be generated or adapted in a given amount of time. Also, the number of iterations per optimization might either be sufficient (albeit accompanied by long planning times) or not be sufficient (for lack of sufficient planning time) and lead to potentially suboptimal plan quality.

In the aforementioned typical round of an optimization loop, a target fluence is mapped to multi-leaf collimator positions and then back to a new current fluence map. This is done on the one hand to update the actual optimization parameters (multi-leaf collimator positions, among others), but also to assure that the next fluence map is actually physically representable by the multi-leaf collimator. This is because the generation of the target fluence maps occurs without information regarding constraints on what kind of fluence maps can be generated using multi-leaf collimators.

To help understand some details set forth herein, it may be helpful to characterize some terminology.

SequencerModule S(m)->MLCs: a module that takes a fluence map and sequences it to multi-leaf collimator positions. This process can be deterministic (or pseudo random, when using a given random seed to initialize a random number generator. This process can be taken also as input to seed multi-leaf collimator positions to guide the process to seek a solution around the seed positions, but this is conceptually not required.

FluenceCalcModule F(MLCs)->m′: This module maps multi-leaf collimator positions and movements back to a fluence map.

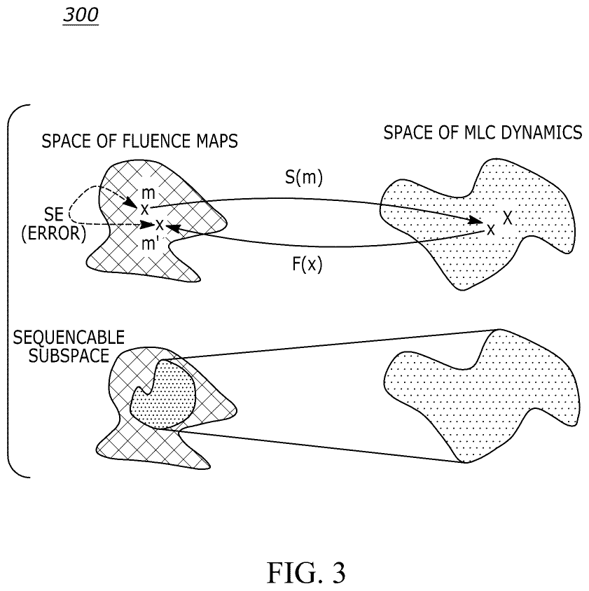

SequencingError SE(m)=|F(S(m))-m|: This module determines how different m′ is when going to multi-leaf collimators and back to fluence. The smaller the sequencing error, the more sequencable the solution becomes. There are multiple choices that could be used in the error norm. For example, simple L1 could be used. It is also often considered that the second gradient of the objective function (such as the Hessian matrix or first diagonal) can be used to normalize individual pixel errors before summing them together. FIG. 3 presents an illustration 300 that depicts these different subspaces and how the foregoing operations map within that space.

Presuming continued use of the foregoing terminology, the operations target->MLCs->new current fluence map corresponds to the operation Proj(t)=F(S(t))=c where t is the target map and c is the new current map. Since the input and output of this combined operation is in the fluence map domain, the applicant has determined that one can interpret the operation as a projection of the target map to a map that is close to the target but which has a low or zero SequencingError SE(m). Therefore, there is a projection going onto or in the direction of a sequencable subspace of the space of possible fluence maps. Sequencability is a property of a fluence map that relates to SequencingError. SE(m)=0 means the map is in the subspace. If the sequencing error is larger than zero, the value describe how far the corresponding fluence map is from the subspace (accordingly, a smaller error means the fluence map is closer to the subspace) (The applicant is using the idea of “projection” here in a somewhat loose sense-a strict independent behavior is not required (for example, there is no sense that applying a projection operation more than once will differ from using it only once.)

The applicant's teachings provide for training an artificial intelligence model that is performing the foregoing projection operation, thereby replacing the sequencing and fluence map generation within a single module. This neural network P(t)~F(S(t)) can be trained by providing many input/output pairs and trained using, for example, L1 Loss. These teachings will accommodate generating these pairs in any of a variety of ways. Optimization trajectories of traditional settings can provide a record of targets and new current fluence maps. Also, for many multi-leaf collimator configurations, corresponding fluence maps m_i can be generated. For an arbitrary number of random fluence maps, the closest sequencable map m_i can be recorded thereby generating an input/output pair for training. By one approach, and depending upon the norm used in SE(m), some additional information can also be given as input for the model (such as the Hessian matrix diagonal).

Any combination or sequence of the foregoing data generation schemes can be used to train such a model. Other ways consistent with the state of the art of training neural networks are also possible and should not be considered as being excluded from these teachings.

As an illustrative example, then, an optimization loop configured in accordance with these teachings can therefore be expressed as follows:
(A) Current Fluence Maps c
(B) Compute Dose based on c
(C) Compute Dose Objectives and the (approximate) derivative of the objective function with respect to the fluence map(s)
(D) Create new target fluence map(s) t based on the derivative and previous map(s)
(E) Compute c′=P(t)
(F)Repeat

After the optimization has converged sufficiently (or prior to such convergence with whatever incremental usage may be desired), the previous Proj(t)=F(S(t)) may also be run. After optimization this information can be used to obtain the final multi-leaf collimator positions.

The applicant has determined that these teachings will accommodate parameterizing fluence maps in different ways. As one example, and assuming that the particle flux is entirely focal (coming from a single point-like source), the fluence maps can be two-dimensional where they are defined on a single plane. In the case where the fluence can be described as being off-focal radiation, they can be three-dimensional and defined in space. Furthermore, each pixel or voxel of the fluence map can have an intensity, particle count, or a whole spectrum associated there with.

In some application settings, sequencing might require more complex inputs than a single fluence map. Sequencing may need to account for the motion between time-points and associated machine limits. Therefore, by one approach, sequencing of an individual field might take into consideration the fluence map associated with the previous and next field(s).

Also, it is possible that the sequencer module is not only considering the reproducibility of the target fluence but also some multi-leaf collimator position-related metrics can be used to define the subspace of possible fluence maps. In practice this sub-space can be redefined more restrictively by requesting that only certain leaf motions are allowed (for example, by limiting the distance one or more leaves can move between control points, or what is the total treatment time). In a setting where the original sequencing module will benefit from a more complex input, the corresponding neural network model can also have corresponding more-complex inputs.

Similarly, if the FluenceCalcModule has additional inputs, these can also be inputs for the neural network model. Generally speaking, any of a variety of additional inputs can also be incorporated during training of the model. For example, the FluenceCalcModule or the Sequencer might depend on the machine type. A specific model for a single machine type can be trained where the input of the machine type is implied. Similarly, a complex input of a machine type can be converted to a much smaller index into a limited amount of different type descriptions. This approach would allow incorporating multiple machine types while conditioning the model to still be able to select between different configurations.

So configured, radiation treatment planning via iterized optimization can be computed very quickly, thereby speeding up the optimization loop (depending upon circumstances, perhaps by a factor of two, though smaller or greater factors are likely possible as well). Furthermore, the training data for the network can be obtained using very high quality, run-time inefficient but precise Sequencing and FluenceMap generators. This is because during the optimization itself only the network inference time is being used. This can lead to both higher speed and better projection, therefore faster convergence and/or better optimization results.

Further aspects of these teachings are provided by the subject matter of the following clauses (where it will be understood that any of these clauses can be combined with any one of more of the other clauses as desired).

Clause 1. A method for optimizing a radiation treatment plan for a particular patient using a particular radiation treatment apparatus, the radiation treatment plan comprising a control point sequence, the method comprising: by a control circuit: accessing at least one objective function to be optimized via an iterative optimization process comprising a plurality of iteration rounds; in at least some of the iteration rounds, employing a neural network that outputs at least one corresponding fluence map; outputting an optimized radiation treatment plan.

Clause 2. The method of clause 1 wherein at least some of the iteration rounds each comprise sequencing only a single field.

Clause 3. The method of clause 1 wherein at least some of the iteration rounds each comprise sequencing at least two fields.

Clause 4. The method of clause 1 wherein at least one of the iteration rounds comprises sequencing at least two fields while holding static at least one field.

Clause 5. The method of clause 1 further comprising: training the neural network using a plurality of input/output pairs.

Clause 6. The method of clause 5 wherein the input/output pairs include at least one of: historical optimization trajectory information; automatically generated optimization trajectory information; automatically generated fluence maps and corresponding sequenceable control points.

Clause 7. The method of clause 5 further comprising: generating a plurality of fluence maps; identifying at least one of the plurality of fluence maps having an acceptable level of sequenceability to provide a selected fluence map; using the selected fluence map as a training target for the neural network.

Clause 8. The method of clause 7 wherein the acceptable level of sequenceability corresponds to an objective measure of sequencing error.

Clause 9. The method of clause 1 wherein at least some of the iteration rounds comprise: accessing a current fluence map; computing radiation dose as a function of the current fluence map; computing dose objectives and an at least approximate derivative of the at least one objective function with respect to the current fluence map; generating a new target fluence map as a function of at least one previous fluence map and the at least approximate derivative of the at least one objective function with respect to the current fluence map; generate a new fluence map as a projection of the new target fluence map, wherein the new fluence map serves as the current fluence map in a subsequent iteration round.

Clause 10. The method of clause 1 further comprising: administering therapeutic radiation to the particular patient using the particular radiation treatment apparatus as a function of the optimized radiation treatment plan.

Clause 11. An apparatus for optimizing a radiation treatment plan for a particular patient using a particular radiation treatment apparatus, the radiation treatment plan comprising a control point sequence, the apparatus comprising: a control circuit configured to: access at least one objective function to be optimized via an iterative optimization process comprising a plurality of iteration rounds; in at least some of the iteration rounds, employ a neural network that outputs at least one corresponding fluence map; output an optimized radiation treatment plan.

Clause 12. The apparatus of clause 11 wherein at least some of the iteration rounds each comprise sequencing only a single field.

Clause 13. The apparatus of clause 11 wherein at least some of the iteration rounds each comprise sequencing at least two fields.

Clause 14. The apparatus of clause 11 wherein at least one of the iteration rounds comprises sequencing at least two fields while holding static at least one field.

Clause 15. The apparatus of clause 11 wherein the control circuit is further configured to: train the neural network using a plurality of input/output pairs.

Clause 16. The apparatus of clause 15 wherein the input/output pairs include at least one of: historical optimization trajectory information; automatically generated optimization trajectory information; automatically generated fluence maps and corresponding sequenceable control points.

Clause 17. The apparatus of clause 15 wherein the control circuit is further configured to: generate a plurality of fluence maps; identify at least one of the plurality of fluence maps having a predetermined acceptable level of sequenceability to provide a selected fluence map; use the selected fluence map as a training target for the neural network.

Clause 18. The apparatus of clause 17 wherein the predetermined acceptable level of sequenceability corresponds to an objective measure of sequencing error.

Clause 19. The apparatus of clause 11 wherein at least some of the iteration rounds comprise: accessing a current fluence map; computing radiation dose as a function of the current fluence map; computing dose objectives and an at least approximate derivative of the at least one objective function with respect to the current fluence map; generating a new target fluence map as a function of at least one previous fluence map and the at least approximate derivative of the at least one objective function with respect to the current fluence map; generate a new fluence map as a projection of the new target fluence map, wherein the new fluence map serves as the current fluence map in a subsequent iteration round.

Clause 20. The apparatus of clause 11 wherein the control circuit is further configured to: administer therapeutic radiation to the particular patient using the particular radiation treatment apparatus as a function of the optimized radiation treatment plan.

Clause 21. A non-transitory computer-readable medium for optimizing a radiation treatment plan for a particular patient using a particular radiation treatment apparatus, the radiation treatment plan comprising a control point sequence, the non-transitory computer-readable medium having instructions stored thereon, that when executed on a processor, perform the steps of: accessing at least one objective function to be optimized via an iterative optimization process comprising a plurality of iteration rounds; in at least some of the iteration rounds, employing a neural network that outputs at least one corresponding fluence map; outputting an optimized radiation treatment plan.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A method for optimizing a radiation treatment plan for a particular patient using a particular radiation treatment apparatus, the radiation treatment plan comprising a control point sequence, the method comprising:
by a control circuit:
accessing at least one objective function to be optimized via an iterative optimization process comprising a plurality of iteration rounds;
in at least some of the iteration rounds, employing a neural network that outputs at least one corresponding fluence map;
outputting an optimized radiation treatment plan.

2. The method of claim 1 wherein at least some of the iteration rounds each comprise sequencing only a single field.

3. The method of claim 1 wherein at least some of the iteration rounds each comprise sequencing at least two fields.

4. The method of claim 1 wherein at least one of the iteration rounds comprises sequencing at least two fields while holding static at least one field.

5. The method of claim 1 further comprising:
training the neural network using a plurality of input/output pairs.

6. The method of claim 5 wherein the input/output pairs include at least one of:
historical optimization trajectory information;
automatically generated optimization trajectory information;
automatically generated fluence maps and corresponding sequenceable control points.

7. The method of claim 5 further comprising:
generating a plurality of fluence maps;
identifying at least one of the plurality of fluence maps having an acceptable level of sequenceability to provide a selected fluence map;
using the selected fluence map as a training target for the neural network.

8. The method of claim 7 wherein the acceptable level of sequenceability corresponds to an objective measure of sequencing error.

9. The method of claim 1 wherein at least some of the iteration rounds comprise:
accessing a current fluence map;
computing radiation dose as a function of the current fluence map;
computing dose objectives and an at least approximate derivative of the at least one objective function with respect to the current fluence map;
generating a new target fluence map as a function of at least one previous fluence map and the at least approximate derivative of the at least one objective function with respect to the current fluence map;
generate a new fluence map as a projection of the new target fluence map, wherein the new fluence map serves as the current fluence map in a subsequent iteration round.

10. The method of claim 1 further comprising:
administering therapeutic radiation to the particular patient using the particular radiation treatment apparatus as a function of the optimized radiation treatment plan.

11. An apparatus for optimizing a radiation treatment plan for a particular patient using a particular radiation treatment apparatus, the radiation treatment plan comprising a control point sequence, the apparatus comprising:
a control circuit configured to:
access at least one objective function to be optimized via an iterative optimization process comprising a plurality of iteration rounds;
in at least some of the iteration rounds, employ a neural network that outputs at least one corresponding fluence map;
output an optimized radiation treatment plan.

12. The apparatus of claim 11 wherein at least some of the iteration rounds each comprise sequencing only a single field.

13. The apparatus of claim 11 wherein at least some of the iteration rounds each comprise sequencing at least two fields.

14. The apparatus of claim 11 wherein at least one of the iteration rounds comprises sequencing at least two fields while holding static at least one field.

15. The apparatus of claim 11 wherein the control circuit is further configured to:
train the neural network using a plurality of input/output pairs.

16. The apparatus of claim 15 wherein the input/output pairs include at least one of:
historical optimization trajectory information;
automatically generated optimization trajectory information;
automatically generated fluence maps and corresponding sequenceable control points.

17. The apparatus of claim 15 wherein the control circuit is further configured to:
generate a plurality of fluence maps;
identify at least one of the plurality of fluence maps having a predetermined acceptable level of sequenceability to provide a selected fluence map;
use the selected fluence map as a training target for the neural network.

18. The apparatus of claim 17 wherein the predetermined acceptable level of sequenceability corresponds to an objective measure of sequencing error.

19. The apparatus of claim 11 wherein at least some of the iteration rounds comprise:
accessing a current fluence map;
computing radiation dose as a function of the current fluence map;
computing dose objectives and an at least approximate derivative of the at least one objective function with respect to the current fluence map;
generating a new target fluence map as a function of at least one previous fluence map and the at least approximate derivative of the at least one objective function with respect to the current fluence map;
generate a new fluence map as a projection of the new target fluence map, wherein the new fluence map serves as the current fluence map in a subsequent iteration round.

20. The apparatus of claim 11 wherein the control circuit is further configured to:
administer therapeutic radiation to the particular patient using the particular radiation treatment apparatus as a function of the optimized radiation treatment plan.

21. A non-transitory computer-readable medium for optimizing a radiation treatment plan for a particular patient using a particular radiation treatment apparatus, the radiation treatment plan comprising a control point sequence, the non-transitory computer-readable medium having instructions stored thereon, that when executed on a processor, perform the steps of:
accessing at least one objective function to be optimized via an iterative optimization process comprising a plurality of iteration rounds;
in at least some of the iteration rounds, employing a neural network that outputs at least one corresponding fluence map;
outputting an optimized radiation treatment plan.
